(19) United States
(12) Patent Application Publication
(10) Pub. No.: US 2021/0170068 A1
(43) Pub. Date: Jun. 10, 2021

(54) A biphasic hydrogel formulation and methods of production and use thereof

(71) Applicant: Gelexcell Competence AB, Lund (SE)

(72) Inventors: Elisabeth STHENGEL, Helsingborg (SE); Anette ISRAELSSON, Lund (SE); Johan WENNERHOLM, Lomma (SE)

(21) Appl. No.: 17/267,806

(22) PCT Filed: Aug. 12, 2019
(86) PCT No.: PCT/SE2019/050740

§ 371 Date: Feb. 10, 2021

(30) Foreign Application Priority Data
Aug. 13, 2018 (SE) 1850974-5

(51) Int. Cl. A61L 26/00; A61P 17/04; A61K 33/30; A61F 13/02; A61F 7/02; A61F 15/00; C08J 3/075; C08K 5/09

(52) U.S. Cl. CPC A61L 26/008; A61P 17/04; A61K 33/30; A61F 13/0253; A61F 7/02; A61L 26/0014; A61F 2013/00119; A61F 15/001; C08J 3/075; C08K 5/09; A61F 2007/0285; A61F 2007/0266; A61F 2007/0244; A61L 26/0066

(57) ABSTRACT

The present invention provides a biphasic formulation, comprising a liquid layer on the outside and an elastic hydrogel wherein the water formed on the surface of the elastic gel is physically cooling the skin by evaporating and to give a first boost of the active ingredients directly when placing on the skin of humans or animals. The biphasic formulation according to the present invention may be arranged in a hydrogel patch which creates an environment that relieves or promotes the healing process for the treatment of insect bites, sunburn, erythema, pruritus, acne, dry skin or callus. In accordance with the present invention, there is provided a biphasic hydrogel composition, comprising 6% or less PVA, physical crosslinking, glycerol as humectant and the use of water impermeable packaging to reach equilibrium of the syneresis and preserve the moisture of the gel during long-term storage.

### TECHNICAL FIELD

The present invention provides a novel biphasic hydrogel composition, methods of production thereof and the use of the composition to treat various dermal conditions.

### TECHNICAL BACKGROUND

Hydrogels are polymer networks extensively swollen with water. They can be prepared using a wide array of natural or synthetic polymers. Due to its physical properties and excellent biocompatibility, polyvinyl alcohol (PVA) is suitable for use in topical hydrogels.

Various polyvinyl alcohol (PVA) based hydrogel formulations are known. Peppas in “Turbidimetric studies of aqueous poly(vinyl alcohol) solutions, Volume 176, Issue 11, November 1975, Pages 3433-3440” was the first to use freeze-thawing as a means for physical crosslinking of the PVA gel, thereby avoiding the use of reactive and potentially irritating chemical crosslinking agents. A sequence of freezing/thawing cycles produce PVA hydrogels with crystallites induced by H-bonds as crosslinking points.

It is well known that the gelation is dependent upon the degree of saponification. Bulky Acetyl groups in the PVA chain prevents the growth of crystals. Based on this a PVA grade with a saponification of 98-100 mol % is usually selected. The gel properties can also be modified by varying polymer concentration, temperature, and the freezing and thawing cycle times.

All hydrogels undergo syneresis to some degree, where water is creeping out of the gel surface. The prior art in this field strives to prevent or to achieve a limited to moderate amount of syneresis.

EP0516026A1 avoids syneresis by adding moisture-absorbing substances in the formulation. Others such as patent document EP0583170 A include the use of crosslinkers or complexing agents such as polyacrylic acid and patent document WO2010029517A1 includes a PVA-acrylamide co-polymer, thereby preventing syneresis.

The disadvantage of using several polymers or complexing agents is the risk of uneven distribution of the additives, or complicated and expensive manufacturing methods. The additives might also be reactive and result in a gel that needs to be extensively washed to remove traces of unreacted crosslinkers before skin contact. U.S. Pat. No. 6,039,977 discloses the use of a monopolymeric hydrogel containing high amounts (>15%) of PVA so that the gel is dry on the surface and syneresis does not occur.

In patent document U.S. Pat. No. 4,734,097A, a monopolymeric hydrogel is described which contains not less than 6 wt. % of a polyvinyl alcohol having a degree of hydrolysis not less than 97%, however, unlike in the present invention, an extra process step, consisting of dehydrating and rehydrating the gel is disclosed. This results in a more complex manufacturing process and a gel with minimal syneresis.

Furthermore, in WO99/29348 there is disclosed pharmaceutical hydrogel formulations containing polyvinyl alcohol. The formulations may serve as drug reservoirs in electro transport drug delivery systems or passive transdermal systems, or they may be used in a variety of other types of dosage forms. Furthermore, WO99/29348 is said to be directed to a method for substantially eliminating syneresis in a polyvinyl alcohol system.

The present invention is directed to providing a hydrogel that is smooth and moist to give a cooling and hydrating effect on the skin, especially in relation to the provision of relieve of pruritus after insect bites.

### SUMMARY OF THE INVENTION

The object presented above is obtained by a dermal device comprising a biphasic hydrogel with one liquid layer and one elastic hydrogel layer, wherein the biphasic hydrogel comprises a monopolymeric formulation, and wherein the dermal device is arranged to provide syneresis, (e.g. measured by determining the weight difference of the gel phase at day 0 and day 14) of at least 10% within 2 weeks. Preferably, the dermal device according to the present invention provides syneresis of at least 15% within 2 weeks. Moreover, according to one specific embodiment of the present invention there is provided syneresis of at least 20% within 2 weeks. Furthermore, according to yet another embodiment of the present invention, the level of syneresis of at least 10% within 2 weeks is determined by measuring the weight difference of the gel phase at day 0 and day 14.

It should be noted that in comparison to the hydrogel formulations disclosed in WO99/29348, which are intended and arranged to substantially eliminate syneresis, the present invention provided a dermal device in which the syneresis is increased in comparison to known hydrogels. This is further described below and also shown in the comparative examples.

Moreover, there are also other important differences when comparing the present invention with the gels of WO99/29348. One example is the fact that the hydrogel according to the present invention is self-supporting, however the devices according to WO99/29348 comprises a backing layer. Some other differences will become apparent from the description below.

The present invention provides a biphasic formulation comprising a liquid layer on the outside and an elastic hydrogel on the inside. The water formed on the surface of the elastic gel is advantageous for physically cooling the skin by evaporating and for creating an environment that promotes the healing process for the treatment of insect bites, sunburn, erythema, pruritus, acne, dry skin or callus.

The present invention also relates to the production of a biphasic hydrogel comprising a liquid layer on the outside and an elastic hydrogel on the inside, to be placed on the skin of humans or animals. The gel is enclosed in a barrier material with low water vapor transmission rate to preserve the moisture until use.

Furthermore, the hydrogel according to the present invention may be provided in a hydrogel patch creating an environment that relieves or promotes the healing process for the treatment of insect bites, sunburn, erythema, pruritus, acne, dry skin or callus. Optionally, the surface of the gel can be increased by introducing pores or ridges, thereby increasing the amount of water retained on the surface of the gel and facilitating its evaporation. The pores are also rendering the gel more elastic and flexible so that it adheres better to the skin.

In relation to the above it should be mentioned that the expression “hydrogel” implies the actual formulation according to the present invention. The expression “patch”, however, refers to a physical product comprising the solidified hydrogel according to the present invention, and includes other alternatives such as a sheet, pad, plaster or other forms or shapes of the self-supported hydrogel.

Contrary to prior art, the present invention is deliberately formulated so that syneresis occurs, causing liquid to creep out of the surface until equilibrium is reached, after one to two weeks. The syneresis with an equilibrium results in a biphasic hydrogel with one liquid layer and one elastic hydrogel layer that is smooth and moist to give the optimized effect on the skin.

To give yet some further examples of prior art documents, the following may be mentioned.

In EP0392845 there is disclosed a method of preparing a solid gel external drug delivery system comprising combining a natural gum or polyvinyl alcohol suspension and a drug that is oil soluble and water insoluble or sparingly water soluble and gelling using an inorganic gelling means. Moreover, EP0392845 also relates to a two-part kit for preparing a solid gel external drug delivery system that comprises an emulsion comprising a natural gum or polyvinyl alcohol suspension and a drug that is oil soluble and water insoluble or sparingly water soluble, and a liquid inorganic gelling means adapted to gel the emulsion of part. Contrary to the present invention, only a limited amount of syneresis is desired according to this patent document, most preferably below 10%.

Furthermore, in WO01/01950 there is described a pre-formed, gel sheet device which is a patch or mask for delivering benefit agents to the skin, hair or nails, comprising from about 30% to about 99.5% of water and a mixture of at least two water-soluble polymeric gel forming agents, wherein the gel comprising the device has an exudate release of greater than 0.7 grams and less than 1.3 grams, a percentage compression at rupture of greater than 45% and less than 90%, Contrary to the present invention, the disclosed formulation consists of at least two gel forming agents, preferably polysaccharides, the gel is inelastic and it breaks when compressed 90% or less. Furthermore, the syneresis is not measured during storage but is measured by pressing the water out one day after manufacturing.

Furthermore, in RU2438654C1 there is provided a hydrogel composition containing 2-allyloxyethanol, lidocaine and/or deoxynate for treating burns wounds and insect bites by pharmacological means. This gel is not self supporting but requires support and strengthening by a solid substrate.

Moreover, in WO2010/029517 there is disclosed a cryogel-forming vinyl alcohol co-polymer is operable to form a cryogel, i.e., a hydrogel formed by crytropic gelation, in an aqueous solution at a concentration of less than about 10% by weight, in the absence of a chemical cross-linking agent and in the absence of an emulsifier. In one embodiment, a vinyl alcohol co-polymer cryogel comprises at least about 75% by weight water and a vinyl alcohol co-polymer, wherein the vinyl alcohol co-polymer is operable to form a cryogel in an aqueous solution at a concentration of less than about 10% by weight, in the absence of a chemical cross-linking agent and in the absence of an emulsifier. The vinyl alcohol co-polymer cryogels are said to be used in various applications including biomedical implants and thin films and for delivery of therapeutic or cosmetic agents.

As may be understood from above, none of the documents above disclose a monopolymeric hydrogel patch, providing an increased syneresis. Although there exist several hydrogels on the market today, and where some of them are intended to be used on the skin, such as mentioned above in WO01/01950, the present invention provides a novel and unique combination of a liquid layer and an elastic hydrogel layer to form a biphasic hydrogel promoting increased syneresis.

Furthermore, none of the documents above disclose a biphasic hydrogel patch to be used for relieving pruritus caused by insect bites by non-pharmacological means for example by providing a cooling effect by the evaporation of water and at the same time protection of the bite from scratching by placement of a protective gel patch.

### EMBODIMENTS OF THE INVENTION

Below specific embodiments of the present invention are disclosed and discussed.

According to one specific embodiment of the present invention, the biphasic hydrogel is physically crosslinked. This is yet another clear difference when comparing the present invention with known hydrogels relevant in this context, which instead use crosslinkers and/or copolymers to enable a binding between the phases in those hydrogels. The crosslinking renders the hydrogel soft but self-supporting and elastic. In this context it may be mentioned that according to one embodiment of the present invention the hydrogel has a force to break above 0.1 N up to 3 N and an elongation to break above 50%.

Yet another difference relates to the concentration of the gel forming polymer. According to one embodiment of the present invention, the dermal device comprises a monopolymeric formulation with a gel forming polymer concentration of maximum 10 wt %, preferably maximum 6 wt %. This low concentration is a clear advantage when comparing with other hydrogels relevant in this context.

The dermal device may comprise different types of gel forming polymers, however polyvinyl alcohol (PVA) is a preferred alternative.

In addition to the gel forming polymer, the dermal device according to the present invention may also comprise other excipients. According to specific embodiment of the present invention, the dermal device also comprises one or more skin humectants, essential oils, plasticizers, antipruritic agents, local anesthetics, debriding agents or cooling agents, or a combination thereof.

To give one example, the present invention provides a monoopolymeric, biphasic hydrogel composition, comprising 10% or less PVA, e.g. below 6% PVA, physical crosslinking, glycerol as humectant and/or plasticizer. Suitably, the biphasic hydrogel composition is used in combination with a packaging with a barrier to preserve the moisture of the hydrogel during long-term storage, e.g. at least 6 months.

Advantageously, depending on use, additional ingredients with beneficial action on various skin conditions may be added, for example, aloe vera, vitamins, calamine, astringents, alcohols, essential oils, cooling agents. Specific examples thereof are presented below.

According to yet another specific embodiment, the dermal device also comprises a basic excipient, for example sodium bicarbonate or ammonia or an acidic excipient, for example acetic acid, citric acid or α-hydroxy acids, providing a pH adjustment to a value that is lower or higher than the normal dermal pH. This change in pH is of relevance in relation to the provision of an increased effect of providing relieve of pruritus after insect bites. Moreover, a decreased pH value may also function as a peeling aid, for the removal of dead skin or callus, in a hydrogel patch according to the present invention.

According to one embodiment, the basic or acidic agent ensures to maintain a pH in the dermal device below 3 or above 7.5. As understood from above, the dermal device according to the present invention may be provided in the form of a patch or the like. According to one specific embodiment of the present invention there is provided a patch, e.g. an insect bite relieving patch, comprising the biphasic hydrogel and where the pH value is kept below 3. To provide this pH value, acetic acid may be used. Therefore, according to one specific embodiment of the present invention, the dermal device comprises acetic acid.

Other alternatives, when a high pH value is intended, i.e. above 8, then ammonia, calamine lotion, or sodium bicarbonate may be used. Other alternatives and more specification are further given below.

Even if the dermal device according to the present invention has a clear focus towards usage as a relieve of pruritus, e.g. after insect bites, the dermal device also finds other use. According to one other alternative, the dermal device may be used to remove or soften callus. In such a case, then the dermal device suitably comprises keratolytic or debriding agents such as acetic acid, salicylic acid, trypsin, collagenase, papain, bromelain or ficin. According to yet another type of application, relieve of burn injury may be mentioned. In such a case, the dermal device may be combined with soothing and/or cooling agents such as menthol or menthol derivates, aloe vera, calamine, eucalyptus or peppermint. Moreover, the dermal device according to the present invention may be used to treat acne, and then the dermal device may comprise topical acne agents such as retinol, essential/ethereal oil, calamine, salicylic acid, benzoe peroxide.

To give yet some other examples in this context, the dermal device according to the present invention may comprise 1-10 wt % glycerol, Sorbitol, propylene glycol or PEG where the ingredient has the function of a skin humectant, and/or plasticizer of the gel and where the hygroscopic properties bind water to the gel.

Regardless of the application direction, the dermal device according to the present invention may provide a cooling effect caused by water evaporation suitable for relieving pruritus. Moreover, the dermal device also functions as a physical barrier providing protection against scratching. This further implies that the dermal device according to the present invention provides a physical effect instead of a pharmacological effect as the case in most existing devices today.

As may be understood from above, the present invention is especially directed to use for relieving of itching from insect bites. In this context, according to one specific embodiment of the present invention, there is provided a dermal device intended as an aid for relieve of itching from insect bites, and said dermal device comprising polyvinyl alcohol (PVA) in the monopolymeric formulation and in a concentration of maximum 10 wt %, said dermal device also comprising acetic acid in a concentration of 1-3 wt %.

To give further examples, the dermal device according to the present invention, when intended to be used to relieve itching from insect bites or other causes, it may comprise antipruritic agents such as: pH adjusting agent for instance 1-3% ammonia or 1-3% acetic acid, 1-10% calamine lotion, 0.1-5% CaCO₃, antihistamine, corticosteroids, xylocaine or other local anesthetics. It should be said, the dermal device, in addition to the antipruritic agents, in itself is also relieving pruritus by the cooling effect caused by water evaporation and by protecting the application area from scratching by providing a physical barrier. In this application, calamine lotion may be of specific relevance. Other possible additives are essential/ethereal oils, e.g. clove oils, tea tree oil and lavender extract. It should be noted that these additives may also be of relevance to be used in a dermal device according to the present invention when being intended for other applications, such as for removal or softening of callus, relieve of burn injury and for treatment of acne.

The dermal device according to the present invention may also be provided with additional physical properties. According to one specific embodiment of the present invention, the elastic hydrogel layer has a non-flat surface to increase the specific surface area thereof and/or to retain a liquid layer against the skin of the user.

Furthermore, and as shortly said above, another difference when comparing the hydrogel according to the present invention with e.g. WO99/29348 is the fact that the hydrogel according to the present invention is self-supportive. In this context it may be mentioned that according to one embodiment of the present invention, the biphasic hydrogel is self-supportive and sticks to the surface of a skin without the need for adhesives.

Moreover, according to yet another embodiment, the non-flat surface comprises surface holes, is fluted or comprises surface extensions to increase the specific surface area and/or to retain a liquid layer against the skin of the user. The intention of this surface effect is to provide a better retention of the liquid layer against the skin and/or to increase the syneresis and/or to facilitate evaporation.

Moreover, according to yet another specific embodiment of the present invention, the dermal device is packed in a barrier material having a cavity for accommodation of the hydrogel to preserve the moisture until use and to retain liquid that is creeping out from the hydrogel during syneresis, and wherein the equilibrium between the gel and liquid form is preserved by packaging the dermal device in a protective barrier until use.

In relation to the present invention, the expression “barrier material” should be interpreted as a water barrier ensuring that water is retained. This also implies that the barrier material according to the present invention is sufficiently permeable or more or less water impermeable, which in this context should be understood to mean any material that will be sufficiently tight against diffusion by evaporation of the actual liquid medium for a period exceeding the recommended shelf life time which would be up to 2 years, typically 12 months.

Therefore, according to one embodiment the present invention provides a biphasic hydrogel packed in a water impermeable material and having a cavity for accommodation of a hydrogel creating an environment to create equilibrium of syneresis which creates a cooling/soothing hydrogel ready to use when the packaging is opened. According to one embodiment, the package as a whole is made of water impermeable material. The package may also be regarded as functioning as a casting mold. In such cases, a patch or plaster according to the present invention obtains the same shape as the package being used. For instance, the cavity may contain grooves or small elevations that molds the hydrogel to achieve an increased surface and ridges or holes are formed.

There present invention also provides a method for the production of a dermal device according to the present invention, said method comprising providing a water-soluble gel forming polymer and water as a solvent;

dissolving the gel forming polymer in water during mixing and heating;
optionally, adding one or more excipients;
mixing to obtain a homogenous solution; and
physically crosslinking and thus solidifying the homogenous solution to a self-supporting hydrogel and storing the hydrogel in a package until syneresis has occurred to create a biphasic hydrogel.

It should be noted that the biphasic hydrogel according to the present invention is obtained after solidification via crosslinking and syneresis. When syneresis has reached an equilibrium, the gel has a moist surface and is suitable to use.

According to one embodiment of the present invention, the monopolymeric formulation is provided with a gel forming polymer concentration in a range of 3-6 wt % and is dissolved in 60-97 wt % water.

Moreover, as mentioned above, the step of filling is suitably performed, before or after the crosslinking step, into a barrier material, such as blister or pouches, having a cavity for accommodation of the hydrogel to preserve the moisture until use and to retain liquid that is creeping out from the hydrogel during syneresis.

Furthermore, according to one specific embodiment the step of physically crosslinking the hydrogel may comprise repeating freezing and thawing cycles or applying gamma radiation.

Moreover, and as mentioned above, the method may also comprise a step of affecting the elastic hydrogel layer to provide a non-flat surface thereof and to increase the specific surface area and/or to enabling to retain a liquid layer against the skin of the user. Furthermore, the patch might be covered with a non-woven adhesive for protection or to enable wearing the patch longer periods.

Further benefits and advantages of the present invention, both in relation to the product, method and use thereof, will become apparent from the examples presented below.

### DESCRIPTION OF THE DRAWINGS

FIG. 1 shows one example of a biphasic hydrogel according to the present invention, where the figure shows a section of the solid gel with a liquid layer surrounding the gel.

FIGS. 2a and 2b show a circular hydrogel according to the present invention, suitably with a thickness of 0.5 to 3 mm. This is one possible example of a shape, but other may of course be used.

FIGS. 3a and 3b shows a hydrogel with ridges, creating an increased surface area.

FIGS. 4a and 4b shows a hydrogel with circular perforations, thereby creating pores to retain the exudate and to allow for evaporation.

The hydrogel according to the present invention may be packaged in different ways. In FIGS. 5a and 5b two different alternatives are shown. In FIG. 5a there is shown a tear open package and in FIG. 5b there is shown a corresponding peel off alternative. Many other package alternatives are possible according to the present invention.

FIG. 6 shows s a graph of the syneresis rate that is slowing down after 14 days. Freshly prepared patches were weighed before packaging in a barrier package. Patches were then taken out of the package and weighed after 1 day, 2 days, 4 days, 11 days and 30 days. Excess fluid was removed before weighing. The amount of syneresis was calculated by subtracting the weight recorded after storage from the weight that was recorded at day 0.

FIG. 7 shows a comparative example referring to syneresis.

FIG. 8 shows a comparative example referring to elongation to rupture.

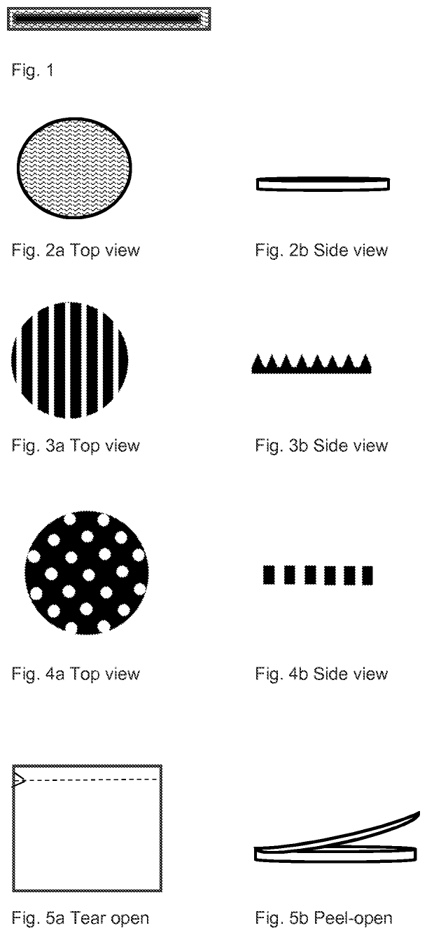

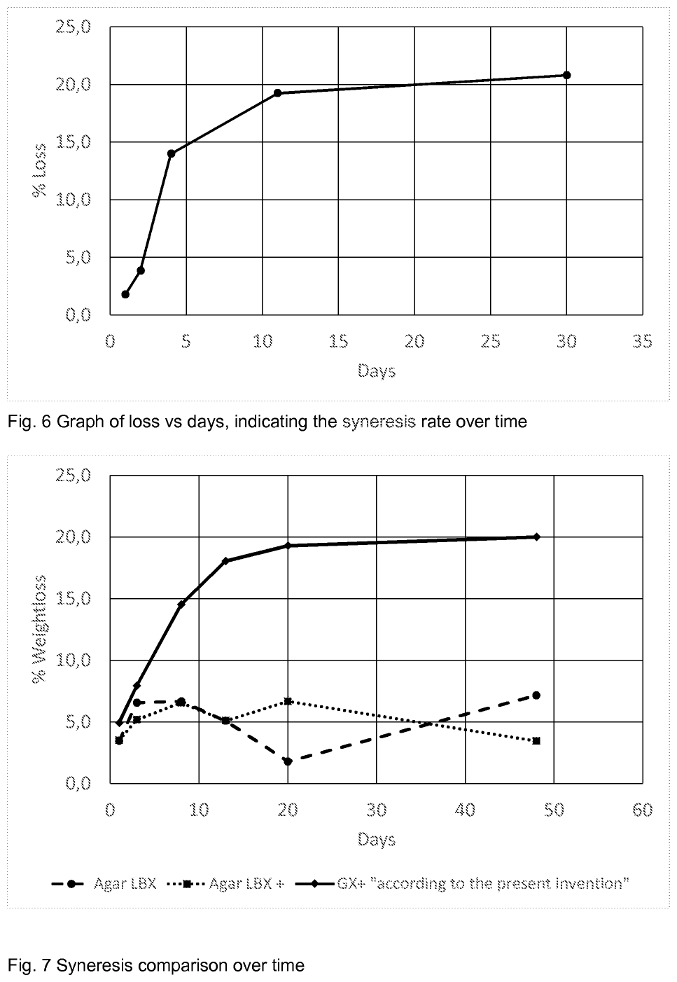

### EXAMPLES

### Example 1: Preparation of PVA Solution

The PVA solution was prepared by dissolving 4-10% wt of PVA in water while heating at 90° C. and gentle stirring until dissolved, (2-6 hours). A preferred PVA average molecular weight range was chosen which resulted in a viscosity of 20-35 mpa of a 4% aqueous solution at 20° C. and which was 97-100% hydrolysed. The PVA used in the experiments was purchased from Cururay Poval and Merck.

### Example 2: Preparation of Acidic Gel for Alleviation Pruritus Caused by Insect Bites or Rashes

A gel intended to alleviate insect bites was prepared by adding glycerol (0-10%) and acetic acid (1-3%) to the PVA solution prepared in example 1 and was physically crosslinked and packaged as in example 7-9 below. Before arriving to this solution, a large number of natural gels evaluated (alginate, gelatin, carrageenan, xanthan and locust bean gum) were evaluated. It was discovered that these polymers did not result in an acceptable hydrogel at acidic pH. When the gel was placed on itching insect bites, the gel had a soothing effect, alleviated pruritus and formed a physical barrier to protect the skin from being scratched thereby protecting the skin from infection and damage caused by scratching the skin.

### Example 3: Advantage of Syneresis

In one experiment, the water loss from gel patches prepared as described in experiment 2 was determined. It was unexpectedly observed that an equilibrium occurred already after 10 days, when about 25% of the liquid had been creeping out of the gel. The hydrogel then remained stable inside the package, during at least 6 months. The liquid phase forming on the outside of the patch is advantageous since it instantly humidifies the skin and results in a cooling/soothing sensation when placed on the skin due to the evaporation of water. The surface layer also ensures that the gel patch does not stick to the package but easily slips out.

### Example 4: Preparation of Hydrogel to Sooth Acne or Other Irritated Skin Conditions

Calamine lotion (2-10%) and/or tee-tree oil (0.1-1%) and/or glycerol and/or vitamin A, was added to the PVA solution prepared in example 1, and packaged and crosslinked as in example 7-9 below. The calamine lotion used in these experiments were purchased from ACO hud AB (“soothing cooling balm”) The ferric oxide in the calamine lotion resulted in an appealing light opaque pink color, the patches were moist and slippery and gave a cooling and soothing effect when placed on irritated skin.

### Example 5: Preparation of a Hydrogel to Remove or Soften Callus or Warts

Glycerol (2-10%) and keratolytic agents such as 0.1 to 2% wt of a suitable protease, or 0.5-to 3% of an acid such as acetic acid or salicylic acid was added to the PVA solution prepared in example 1 and crosslinked and packaged as in example 6-8 below. A gel prepared as described in this example, containing glycerol as humectant and trypsin as the keratolytic enzyme was found to be comfortable to wear and softened the skin.

### Example 6: Packaging Hydrogel after Freeze/Thaw

The solution was prepared as in example 1, 4, or 5 and dispensed in to molds of the desired shape. The molds were exposed to repeated freezing and thawing of 6 to 12 hours for 2 to 5 times (2-5×6-12 hours) until the gel was sufficiently solidified (Force to break >0.1 and <3 N). The crosslinked and solidified hydrogel patches were removed from the molds and packed in aluminum pouches or plastic pouches and sealed (FIGS. 3a and 3b).

### Example 7: Filling in Blisters Before Freeze/Thaw

PVA solutions were prepared as in example 1, 4, or 5 and dispensed into cavities in blister packages prepared in the desired shape, while in liquid form, sealed and then exposed to freeze-thawing cycles until solidified.

### Example 8: Filling in Sachets Before Freeze/Thaw

PVA solutions were prepared as in example 1, 4, or 5 and dispensed into pouches of the desired form that were sealed manually or in a vertical or horizontal form/fill/seal machine, and then exposed to freeze-thawing cycles until solidified.

### Example 9: Protecting the Applied Gel with a Nonwoven Layer

In one example the hydrogels are combined with pre-cut pieces of thin breathable non-woven material with adhesive. A material from 3M art #1776 was found to be suitable to protect and keep the patch in place while still allowing for water to evaporate

### Example 10: Preparation of a Stable, Elastic Hydrogel Patch with a Syneresis at Equilibrium of at Least 20%

The desired amount of syneresis and elasticity is reached by using a PVA polymer that is >97% hydrolyzed with a molecular weight range of 90 000-100 000 g/mol, keeping the final PVA concentration in the formulation below 8%, temperature cycling a maximum of 4 times, and packaging in a sachet or blister with a sufficiently high water barrier. The result is a highly elastic gel with a 10-20% syneresis after 2 weeks, that will be elastic and moist for at least 6 months.

### Comparative Experiments

The following materials were used to conduct a comparative trial of a PVA gel according to the present invention, with other gels (Table 1):

TABLE 1
Composition of gels used for comparing
syneresis and elongation to break

| | | Agar 1 | Agar 2 | Agar 3 | Agar 4 |
|---|---|---|---|---|---|
| | PVA Gel* | (Agar | (Agar | (Agar | (Agar |
| Material | (GX⁺) | LBX⁺) | LBX) | XC) | XC⁺) |
| PVA | 6% | — | — | — | |
| Agar | — | 0.9% | 0.9% | 0.6% | 0.6% |
| Glycerol | 7.5% | 15% | 15% | 10% | 10% |
| Acetic Acid | 2% | 2% | — | — | 2% |
| Locust Bean Gum | — | 0.1% | 0.1% | — | |
| Xhantan Gum | | 0.1% | 0.1% | 0.1% | 0.1% |
| Konjac Mannan | | | | 0.3% | 0.3% |

*According to the present invention
Agar, locust bean gum, konjac mannan and xanthan are first dispersed in glycerol, water is added while stirring, heated until clear.

The following methods were used to compare syneresis:

3 g gels are molded in 3 cm diameter silicone forms. PVA gels were temperature cycled 3 times and agar gels were left to settle overnight. The solidified gels were weighed and packaged in high barrier sachets (PET/PE/ALU/PE);

After 1, 3, 8, 13, 20 and 48 days, excess liquid shaken off and the gels were weighed. Syneresis was calculated by the following formula: (Initial weight−weight after storage)*100/Initial weight

Syneresis equilibrium was evaluated over time and the results are shown in FIG. 7. Here it is seen that a gel according to the present invention has a high syneresis in comparison to the known agar gels.

Furthermore, a manual elongation to break test was also performed. The following procedure was performed:

1) Gels are cut in to 4×1 cm strips;

2) Clamps are attached in both ends with 2 cm gel strip in between* *The polysacharide gels are too brittle to be held by the clamp so they are handheld instead

3) Gels are pulled apart along a steel ruler; and

4) The % elongation at the time when the gel breaks are noted;

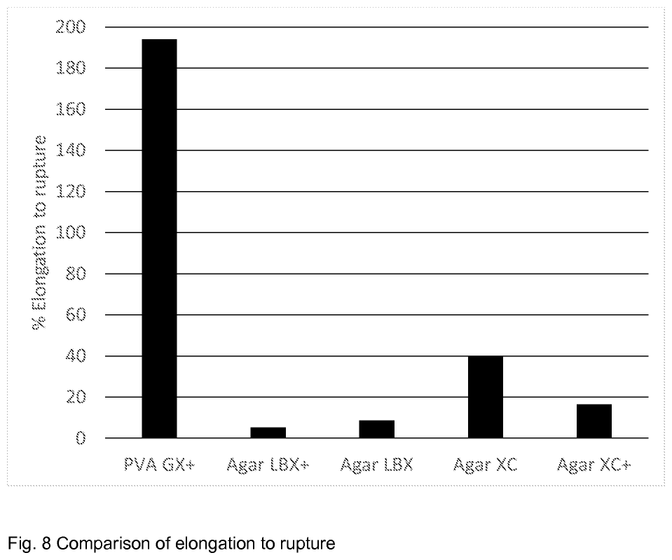

The results are presented in FIG. 8 where it is clear that the gel according to the present invention has a much higher elongation to break (rupture) than known agar gels.

Furthermore, the elasticity of a PVA hydrogel according to the present invention was also compared with 3 different polysaccharide gels, by measuring the force required to rupture the gel.

### Method and Material of the Elasticity Trials

### Gel Preparation for the Elasticity Trials

Gels with the dimensions 2.2 cm diameter and 1.2 mm height were prepared as follows: The polysaccharides were dispersed in glycerol, water added and then heated to 90° C. 4 ml of each gel was pipetted in a 6-well plate while still warm. Left to gel over-night. The recipes were taken from WO0101950A1, pre-formed gel sheet, with the exception that agarose was replaced with agar and that no preservatives were added (see table 2 below).

TABLE 2
Composition of gels used for compression testing

| | | | | Gel 4:1 and 4:2 |
|---|---|---|---|---|
| | Gel 1 | Gel 2 | Gel 3 | (acc. to present |
| Material | (E.G.1) | (E.G.6) | (E.G.8) | invention) * |
| PVA | | | | 6% |
| Acetic Acid | | | | 2% |
| Agar Agar | 0.9% | 0.4% | — | — |
| Locust Bean Gum | 0.1% | — | 0.5% | — |
| Xhantan gum | 0.1% | 0.1% | 0.5% | — |
| Konjac Mannan | 0.2% | 0.2% | — | — |
| Glycerol 99.5% | 15% | 10% | 10% | 7.5% |

*4 ml was pipetted per well. Gel 4:1 was temperature cycled twice and Gel 4:2 was temperature cycled three times

### Compressive Rupture Test

Compressive rupture was measured manually by placing the gels between two glass petri-dishes. The dish was place on a scale and weights were added on top until breakage could be seen.

### Result of the Rupture Test

The PVA gels according to the present invention could not be ruptured within the capacity of the scale and the result for both PVA gels is therefor set to >33N. They were compressed >90% but did not break and sprung back to their original form, when pressure was released.

Gel 1 required 3.8 N to break and Gel 2 1.4N. Gel 3 could not be measured since it did not hold together. The results are presented below in table 3.

TABLE 3
Rupture tests of known gels in comparison
to gels according to the present invention

| Gel | S1 (N) | S2 (N) | mean |
|---|---|---|---|
| 1 | 4 | 3.5 | 3.8 |
| 2 | 1.5 | 1.2 | 1.4 |
| 3 | Could not | Could not | Could not |
| | measure | measure | measure |
| 4:1 according to the | >33 | >33 | >33 |
| present invention | | | |
| 4:2 according to the | >33 | >33 | >33 |
| present invention | | | |

As may be seen, the polysaccharide gels are brittle and breaks easily. Force to rupture is at least 10 times lower compared to the PVA gels according to the present invention.

Based on the above, according to yet another specific embodiment of the present invention, the biphasic hydrogel and thus dermal device is elastic with an elongation to break of at least 100% and the force required to elongate the dermal device 50% is less than 0.5 N. This is of interest so that the dermal device adjusts to irregularities of the skin surface where it is attached. Moreover, this is also a clear difference of the dermal device and hydrogel according to the present invention when comparing to known hydrogels.

1. A dermal device comprising a biphasic hydrogel with one liquid layer and one elastic hydrogel layer, wherein the biphasic hydrogel comprises a monopolymeric formulation, and wherein the dermal device is arranged to provide syneresis of at least 10% within 2 weeks.

2. The dermal device according to claim 1, wherein the biphasic hydrogel is physically crosslinked.

3. The dermal device according to claim 1, wherein the dermal device comprises a monopolymeric formulation with a gel forming polymer concentration of maximum 10 wt %, preferably maximum 6 wt %.

4. The dermal device according to claim 1, wherein the dermal device comprises polyvinyl alcohol (PVA).

5. The dermal device according to claim 1, wherein the dermal device also comprises one or more skin humectants, essential oils, plasticizers, antipruritic agents, local anesthetics, debriding agents or cooling agents, or a combination thereof.

6. The dermal device according to claim 1, wherein the dermal device also comprises a basic or acidic agent providing a pH adjustment to a value that is lower or higher than the normal dermal pH.

7. The dermal device according to claim 6, wherein the basic or acidic agent ensures to maintain a pH in the dermal device below 3 or above 7.5.

8. The dermal device according to claim 6, wherein the dermal device comprises acetic acid.

9. The dermal device according to claim 1, wherein the level of syneresis of at least 10% within 2 weeks is determined by measuring the weight difference of the gel phase at day 0 and day 14.

10. The dermal device according to claim 1, wherein the biphasic hydrogel and thus dermal device is elastic with an elongation to break of at least 100% and the force required to elongate the dermal device 50% is less than 0.5 N.

11. The dermal device according to claim 1, said dermal device providing a cooling effect caused by water evaporation suitable for relieving pruritus and wherein the dermal device also functions as a physical barrier providing protection against scratching.

12. The dermal device according to claim 1, said dermal device intended as an aid for relieve of itching from insect bites, and said dermal device comprising polyvinyl alcohol (PVA) in the monopolymeric formulation and in a concentration of maximum 10 wt %, said dermal device also comprising acetic acid in a concentration of 1-3 wt %.

13. The dermal device according to claim 1, wherein the elastic hydrogel layer has a non-flat surface to increase the specific surface area thereof and/or to retain a liquid layer against the skin of the user.

14. The dermal device according to claim 1, wherein the non-flat surface comprises surface holes, is fluted or comprises surface extensions to increase the specific surface area and/or to retain a liquid layer against the skin of the user.

15. The dermal device according to claim 1, wherein the biphasic hydrogel is self-supportive and sticks to the surface of a skin without the need for adhesives.

16. The dermal device according to claim 1, wherein the dermal device is packed in a barrier material having a cavity for accommodation of the hydrogel to preserve the moisture until use and to retain liquid that is creeping out from the hydrogel during syneresis, and wherein the equilibrium between the gel and liquid form is preserved by packaging the dermal device in a protective barrier until use.

17. A method for the production of a dermal device according to claim 1, said method comprising
providing a water-soluble gel forming polymer and water as a solvent;
dissolving the gel forming polymer in water during mixing and heating;
optionally, adding one or more excipients;
mixing to obtain a homogenous solution; and
physically crosslinking and thus solidifying the homogenous solution to a hydrogel and storing the hydrogel in a package until syneresis has occurred to create a biphasic hydrogel.

18. The method according to claim 17, wherein the monopolymeric formulation is provided with a gel forming polymer concentration in a range of 3-6 wt % and is dissolved in 60-97 wt % water.

19. The method according to claim 17 or 18, wherein the step of filling is performed before or after the crosslinking step, into a barrier material having a cavity for accommodation of the hydrogel to preserve the moisture until use and to retain liquid that is creeping out from the hydrogel during syneresis.

20. The method according to claim 17, wherein the step of physically crosslinking the hydrogel comprises repeating freezing and thawing cycles or applying gamma radiation.

21. The method according to claim 17, wherein said method also comprises a step of affecting the elastic hydrogel layer to provide a non-flat surface thereof and to increase the specific surface area and/or to enabling to retain a liquid layer against the skin of the user.
